(19) United States
(12) Plant Patent Application Publication
(10) Pub. No.: US 2003/0135901 P1
(43) Pub. Date: Jul. 17, 2003

(54) Hybrid tea rose plant named 'Ruimz15'

(76) Inventor: Henk de Groot, De Kwakel (NL)

Correspondence Address: CHRISTIE, PARKER & HALE, LLP, P.O. BOX 7068, PASADENA, CA, 91109-7068, US

(21) Appl. No.: 10/341,893

(22) Filed: Jan. 13, 2003

(30) Foreign Application Priority Data
Jan. 15, 2002 (EP) 2001/2115
May 27, 2002 (EC) 328-02

(51) Int. Cl. A01H 5/00

(52) U.S. Cl. PLT/134

(57) ABSTRACT

A new and distinct hybrid tea rose plant having attractive large cream flowers.

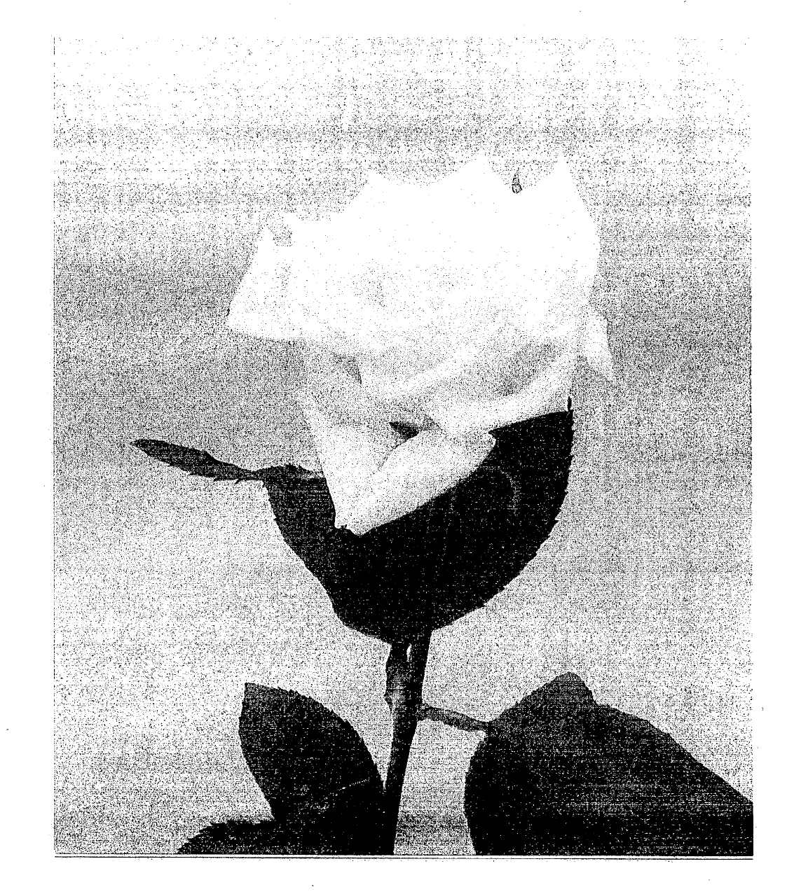

### BACKGROUND OF THE INVENTION

The present invention relates to a new and distinct variety of rose plant of the hybrid tea rose class, which was discovered in a controlled planting of ‘RUILIRO’ (U.S. Plant Pat. No. 11,175) as a natural mutation. The varietal denomination of this new rose is named ‘RUIMZ15’. The new variety was grown from cuttings in The Netherlands.

### SUMMARY OF THE INVENTION

Among the novel characteristics possessed by this new variety which distinguishes it from its parent and all other varieties of which I am aware are its flower color.

Asexual reproduction by propagation of grafting of the new variety as performed in De Kwakel, The Netherlands shows that the foregoing and other distinguishing characteristics come true to form and are established and transmitted through succeeding propagation.

### COMPARISON WITH PARENT

The new variety differs from its parent in flower color, inasmuch as the flowers of ‘RUILIRO’ are a pink blend and flowers of the new variety are cream.

### BRIEF DESCRIPTION OF THE ILLUSTRATION

The accompanying illustration shows a typical specimen of the new variety depicted in color as nearly true as is reasonably possible to make the same in a color illustration of this character.

### DESCRIPTION OF THE NEW VARIETY

The following is a detailed description of the new variety with color terminology in accordance with The Royal Horticultural Society Colour Chart (RHSCC). The terminology used in color description herein refers to plate numbers in the aforementioned color chart. Phenotypic expression may vary with light availability, environmental and cultural conditions.

The following observations are made of 9-month old plants grown in a greenhouse in De Kwakel, The Netherlands during September from a cutting.

In all respects other than characteristics of the flower, plants of the new variety are the same as of ‘RUILIRO’. Accordingly, the description set forth in U.S. Plant Pat. No. 11,175, dated Jan. 11, 2000, the disclosure of which is expressly incorporated herein by reference.

Flower color of ‘RUIMZ15’:

After sepals part and petals unfurl, color of petal top surface near edges is near 8D. Petals undersurface exhibits same coloration as top surface. Color at point of petal attachment is near 2B.

I claim:

1. A new and distinct hybrid tea rose plant of the variety substantially as shown and described.
